SEQUENCE LISTING (5 sequences)

SEQ ID NO: 1 - protein, 6 aa, Artificial Sequence
  binding partner
CCXXCC

SEQ ID NO: 2 - protein, 8 aa, artificial sequence
  flag/anti-flag pair
DYKDDDDK

SEQ ID NO: 3 - protein, 9 aa, artificial sequence
  HA/anti HA pair
YPYDVPFYA

SEQ ID NO: 4 - DNA, 6758 bp, artificial sequence
  plasmid
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctttgagacatggccttaccagtgaccgccttgctcctgccgctggccttgctgctccacgccgccaggccggccgccgctagcggcatcgactacaaggacgacgatgacaaggccggcatcgatgccatcatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggtgatatccagcacagtggcggccgcgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgtcgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 5845 bp, artificial sequence
  plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatcatgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2015/0045253 A1
(43) Pub. Date: Feb. 12, 2015

(54) METHOD FOR DETERMINING THE ABILITY OF AN ANTIBODY TO KEEP CELLS CLOSE TO ONE ANOTHER

(71) Applicant: CISBIO BIOASSAYS, Codolet (FR)

(72) Inventor: Delphine Jaga, Sauveterre (FR)

(21) Appl. No.: 14/386,232

(22) PCT Filed: Mar. 18, 2013
(86) PCT No.: PCT/FR2013/050567

§ 371 Date: Sep. 18, 2014

(30) Foreign Application Priority Data
Mar. 19, 2012 (FR) 1252433

(51) Int. Cl. G01N 33/68

(52) U.S. Cl. CPC G01N 33/6854; G01N 2500/10; G01N 2500/04
USPC 506/9; 506/14

(57) ABSTRACT

The invention relates to a method for determining the ability of a candidate antibody to keep a first cell and a second cell close to one another, said method comprising the following steps:
bringing said candidate antibody in contact with (i) a first cell expressing, in the extracellular portion of its plasma membrane, a first protein that is known to be or is suspected of being recognized by the candidate antibody and (ii) a second cell expressing, in the extracellular portion of the plasma membrane, a second protein that is also known to be or suspected of being recognized by the candidate antibody, each of the proteins being labeled with a member of a pair of FRET partners, and
measurement of the FRET signal and comparison with the signal measured in the absence of the candidate antibody.

The invention also relates to a reagent kit for carrying out this method.

### FIELD OF THE INVENTION

The invention relates to a method for determining the ability of a candidate antibody to keep cells close to one another, and in particular to keep effector cells or cells imitating effector cells bearing a first antigen or a receptor of the crystallizable fragments Fc (hereinafter “Fc receptors” or FcR) close to cells bearing a second target antigen. The invention is particularly useful for evaluating the cytotoxic character of an antibody potentially usable as a therapeutic antibody.

### PRIOR ART

For some years now, monoclonal antibodies have allowed a therapeutic revolution. In addition to the classical antibodies, progress in molecular biology and immunology has led to the development of antibodies possessing variable domains or different paratopes and therefore capable of recognizing different epitopes. Although the clinical efficacy of antibodies no longer requires proof, their mode of action in patients is still relatively poorly understood.

The therapeutic effect of antibodies targeting membrane antigens notably involves the recruitment of effector cells expressing either receptors for the crystallizable fragment of the antibodies (hereinafter, “Fc receptors”), or in the case of bi- or trispecific recombinant antibodies, the recruitment of cells expressing other antigens such as for example the CD3, or CD28 receptors. Antibodies of this last-mentioned type have the advantage of allowing the recruitment of effector cells not possessing Fc receptors.

The Fc receptors are proteins present on the surface of certain cells contributing to the functions of the immune system, in particular NK (“natural killer”) lymphocytes, macrophages, neutrophils and mastocytes. Their name derives from their ability to bind to the Fc (crystallizable fragment) region of the antibodies. There are several types, which are classified according to the type of antibody that they recognize: the Fc gamma receptors (FcγR) bind to the IgGs, the Fc alpha receptor (FcαR) binds to the IgAs and the Fc epsilon receptors (FcεR) bind to the IgEs.

Binding of the Fc receptor to an antibody triggers different mechanisms depending on the nature of the cell on which this receptor is expressed. Table 1 is a summary of the cellular distribution of the various Fc receptors and of the mechanisms triggered by binding of the receptor to an antibody.

TABLE 1

| | Antibody | Cellular | Effet following binding |
|---|---|---|---|
| Receptor | recognized | distribution | to the antibody |
| FcγRI | IgG1 and | Macrophages | phagocytosis |
| (CD64) | IgG3 | Neutrophils | cellular activation |
| | | Eosinophils | generation of reactive |
| | | Dendritic cells | oxygen destruction of |
| | | | microbes |
| FcγRIIA | IgG | Macrophages | phagocytosis |
| (CD32) | | Neutrophils | degranulation |
| | | Eosinophils | (eosinophils) |
| | | Platelets | |
| | | Langerhans cells | |
| FcγRIIB1 | IgG | B lymphocytes | no phagocytosis |
| (CD32) | | Mastocytes | inhibition of the activity |
| | | | of the cells |
| FcγRIIB2 | IgG | Macrophages | phagocytosis |
| (CD32) | | Neutrophils | inhibition of the activity |
| | | Eosinophils | of the cells |
| FcγRIIIA | IgG | NK lymphocytes | induction of antibody- |
| (CD16A) | | Macrophages | dependent cellular |
| | | (certain tissues) | cytotoxicity (ADCC) |
| | | | induction of the release |
| | | | of cytokines by |
| | | | macrophages |
| FcγRIIIB | IgG | Eosinophils | destruction of microbes |
| (CD16b) | | Macrophages | |
| | | Neutrophils | |
| | | Mastocytes | |
| | | Follicular dendritic | |
| | | cells | |
| FcεRI | IgE | Mastocytes | degranulation |
| | | Eosinophils | |
| | | Basophils | |
| | | Langerhans cells | |
| FcεRII | IgE | B lymphocytes | possible adhesion |
| (CD23) | | Eosinophils | molecule |
| | | Langerhans cells | |
| FcαRI | IgA | Monocytes | phagocytosis |
| (CD89) | | Macrophages | destruction of microbes |
| | | Neutrophils | |
| | | Eosinophils | |
| Fcα/μR | IgA and IgM | B lymphocytes | endocytosis |
| | | Mesangial cells | destruction of microbes |
| | | Macrophages | |

Therapeutic antibodies triggering the ADCC (antibody-dependent cellular cytotoxicity) mechanism are currently marketed (Herceptin®, Cetuximab®) and are indicated in the treatment of certain cancers: the Fc fragment of these antibodies binds to an Fc receptor present on the NK lymphocytes, whereas the paratopes of these antibodies recognize an antigen present on tumor cells (Herceptin: Her2, Cetuximab: Her1). Binding of these antibodies both to an NK cell and to a cancer cell notably leads to destruction of the latter by the activation of the ADCC mechanism.

Other therapeutic antibodies whose paratopes are capable of recognizing various antigens, and are therefore capable of recruiting an effector cell independently of the recruitment mediated by their Fc domain, have also been developed. We may mention, among others, blinatumobab capable of binding both to CD19 and CD3, Catumaxomab, which binds to EpCAM and CD3, Hertumaxomab, which recognizes Her3 and CD3, MDX-210 which binds to Her2 and CD64, MDX-447 which is specific for EGFR and CD64 or rM28 which recognizes NG2 and CD28.

It would be particularly advantageous to be able to detect both binding of the antibody to the Fc receptor present on the effector cell and binding to its target antigen, in particular in the context of developing antibodies for diagnostic or therapeutic purposes. In the same way, for the bi- or trispecific antibodies, it would be very useful to be able to detect simultaneous binding of the antibody to its two different antigens.

The techniques currently available for studying the binding of antibodies to the Fc receptors are relatively tedious:

The system marketed under the name Biacore® allows detection of interactions between two binding partners and is based on the phenomenon of surface plasmon resonance. This system requires immobilization of the Fc receptor or else of a fragment of the Fc domain of the antibody tested on a microchip, capable of generating a signal that depends on the refractive index at its surface (Biacore Journal Number 1, 2009, 15-17). This approach has several drawbacks, notably a critical step of fixation of one of the binding partners to the surface of the microchip, the need for expensive equipment that requires a certain expertise, and the fact that the technique does not allow working with living cells expressing the Fc receptor being studied. Moreover, this approach also does not supply information regarding any binding of the antibody tested to its antigen in its cellular conformation.

French patent application FR2844521 describes an approach according to which an antibody is brought into contact with cells expressing the CD16 receptor in the presence of the antigen of said antibody, and at least one cytokine produced by the cells is measured, an increase in the amount of cytokine being representative of the activation of said cells. This technique does not allow direct measurement of the binding of the antibody to the FcR, nor does it supply information regarding binding of said antibody to its antigen. It also requires a step of centrifugation of the supernatant for detecting the cytokines secreted by the cells.

There are several laboratory methods for studying the efficacy of antibodies having potentially a therapeutic effect via a cytotoxic effector cell, in particular an ADCC mechanism. These methods generally consist of bringing the antibodies under investigation into contact with effector cells and target cells, and of detecting the destruction of the target cells by measuring:
the radioactivity released by the target cells (when they have for example been loaded beforehand with chromium-51),
the activity of enzymes of the reductase/dehydrogenase type released in the extracellular matrix, which will modify the spectroscopic properties of compounds added to said matrix (such as XTT, MTS, WST, MU, resazurin),
the activity of the GAPDH enzyme released in the extracellular matrix, which causes production of ATP, and emission of light if luciferase has been introduced into said matrix beforehand (U.S. Pat. No. 6,811,990),
release of europium (when the target cells have previously been loaded with this compound, DELFI A® EuTDA Cytotoxicity ADOl 16 kit (PerkinElmer)),
staining of the dead cells with products of the tryptan blue, propidium iodide type.

These techniques have several drawbacks, notably in terms of convenience of use, sensitivity, specificity (considerable background noise) but the most significant is that they only supply indirect information and do not allow direct and relatively simple quantification of the ability of a candidate antibody to keep two cells close to one another.

The company Cisbio Bioassays markets a product range under the name Tag-lite®, for labeling proteins expressed by cells with fluorescent compounds, as well as FRET-partner fluorescent compounds (HTRF®). As the phenomenon of FRET between an energy donating compound and an acceptor compound is dependent on the distance between these two compounds, the products in the Tag-lite® range make it possible to study molecular interactions in a cellular context. One of the applications of this technique is investigation of the interaction between a receptor coupled to proteins G (RCPG) labeled with a FRET partner, with its ligand labeled with a second FRET partner. This approach makes it possible for example to perform competitive assays for evaluating the binding of new potential ligands of these RCPGs. Another example of application of these products is investigation of the dimerization of RCPGs labeled with FRET partner compounds.

Bacsó et al. (Immunol Lett. 1996 December; 54(2-3): 151-6) determined the efficacy of intercellular FRET between on the one hand the CD8 membrane protein complex of a cytotoxic T lymphocyte (CTL), labeled with an anti-CD8alpha antibody conjugated with FITC, and on the other hand with MHC-I antigens present on a target cell, labeled with a specific antibody of the alpha 2/alpha 3 domains of HLA A, B, C conjugated with TRITC. In the same way, these authors also determined the efficacy of energy transfer between on the one hand an anti-ICAMI antibody conjugated with FITC and binding to target cells, and on the other hand an anti-LFA-I antibody conjugated with TRITC and binding to the CTL. The technique used for measuring the evaluation of FRET consists of measuring the photobleaching of the donor compound (FITC) and requires a microscope. The authors observed FRET at the points of contact between the cells in the first case (CD8/MHCI) but not in the second (ICAMI/LFAI), which in their opinion confirms the knowledge at that time regarding the organization of these molecules in the membrane.

At present there is no method for investigating potentially therapeutic antibodies, supplying, in an experiment, quantifiable information on the ability of these antibodies to simultaneously recognize their target cell and to recruit an effector cell, in conditions relatively close to those in which the candidate antibody will act in vivo.

The inventors have developed a method for solving this problem.

### BRIEF DESCRIPTION OF THE FIGURES

FIG. 1 shows the FRET signal emitted after bringing in contact cells expressing the CD16a receptor on the one hand and cells expressing the HER1 receptor on the other hand, in the presence of various antibodies, the CD16a and HER1 receptors being labeled directly with a terbium cryptate and a red acceptor fluorophore, respectively.

FIG. 2 shows the FRET signal emitted after bringing in contact cells expressing the CD16a receptor on the one hand and cells expressing the HER2 receptor on the other hand, in the presence of various antibodies, the CD16a and HER1 receptors being labeled directly with a terbium cryptate and a red acceptor fluorophore, respectively.

FIG. 3 shows the FRET signal emitted after bringing in contact cells expressing the CD16a receptor on the one hand and cells expressing the HER1 receptor on the other hand, in the presence of various antibodies, the CD16a receptor being labeled directly with a terbium cryptate, and the HER1 receptor being labeled either indirectly via formation of dimers with the HER3 receptor, itself labeled directly with a red acceptor fluorophore, or else by nonspecific labeling of the CXCR4 receptor, which does not form a dimer with HER1 but whose overexpression and homogeneous distribution on the cell surface nevertheless allow observation of a signal.

FIG. 4 shows the plasmid pM7 pTagLite-Snap FcgRIIIa.

FIG. 5 shows the plasmid pM8 wild-type gamma chain pcDNA3.1/Hygro(+).

### DESCRIPTION

The invention is based on the unexpected discovery that it is possible to measure a FRET signal between two cells kept close to one another by an antibody. This discovery is particularly useful for studying antibodies potentially usable as therapeutic antibodies (candidate antibodies) whose mode of action involves the recruitment, or bringing closer together, of several cells, including an effector cell, since it allows simple investigation and quantification, in the course of a single experiment, of the binding of the antibody both to its target cell and to the effector cell, or a cell imitating an effector cell.

“Candidate antibody” means an antibody that has been selected for its ability to bind to a given target antigen expressed for example by a target cell and that is moreover capable of binding to at least one other protein, notably but not only via its Fc fragment or else via one of its paratopes. The term antibody is to be understood here in its broadest sense, and in addition includes conventional antibodies, the compounds consisting partly of at least one F(ab) antibody fragment, at least one F(ab′), at least one single-chain variable fragment (scFv), at least one single-domain antibody (sdAb) and the various combinations of these elements.

The invention thus relates to a method for determining the ability of a candidate antibody to keep a first cell and a second cell close to one another, this method comprising the following steps:
bringing the following elements in contact:
   (i) a first cell expressing, in the extracellular portion of its plasma membrane, a first protein that is known to be or is suspected of being recognized by the candidate antibody, said first protein being labeled directly or indirectly with the first member of a pair of FRET partners,
   (ii) a second cell expressing, in the extracellular portion of the plasma membrane, a second protein that is also known to be or suspected of being recognized by the candidate antibody, said second protein being labeled directly or indirectly with the second member of said pair of FRET partners,
   (iii) said candidate antibody;
measurement of the FRET signal and comparison with the signal measured in the absence of the candidate antibody, an increase in the signal measured in the presence of the candidate antibody relative to that measured in its absence being indicative of the ability of said antibody to keep the first cell and the second cell close to one another.

This novel application of the FRET principle is also particularly unexpected, in that it might have been feared that the distance between the two cells is too great to allow observation of a variation of the FRET signal during formation of the complex comprising the antibody and the two cells.

Moreover, this method makes it possible to obtain a quantifiable value representative of the binding of the candidate antibody to two proteins, and therefore makes it possible to compare two given candidate antibodies: at equal concentration, the antibody leading to the highest FRET signal will have a greater ability to keep the two cells close to one another, which might be reflected in an improved therapeutic effect.

The expression “pair of FRET partners” denotes a pair consisting of an energy-donating fluorescent compound (hereinafter “fluorescent donor compound”) and an energy-accepting compound (hereinafter “acceptor compound”); when they are close to one another and when they are excited at the excitation wavelength of the fluorescent donor compound, these compounds emit a FRET signal. It is known that for two fluorescent compounds to be FRET partners, the emission spectrum of the fluorescent donor compound must partially overlap the excitation spectrum of the acceptor compound.

“FRET signal” means any measurable signal representative of FRET between a fluorescent donor compound and an acceptor compound. A FRET signal may therefore be a change in the intensity or lifetime of luminescence of the fluorescent donor compound or of the acceptor compound when the latter is fluorescent.

The expression “protein recognized by an antibody” is used here in the broadest sense, i.e. either the protein bears an epitope recognized by at least one of the paratopes of the antibody, or the protein has a domain capable of binding to the crystallizable fragment (Fc fragment) of the antibody.

In a particular embodiment, the first cell expresses, in the extracellular portion of its plasma membrane, a first protein that is a membrane receptor of the Fc fragment of the antibodies, labeled directly or indirectly with the first member of a pair of FRET partners, and the second cell expresses, in the extracellular portion of its plasma membrane, a second protein that bears an epitope recognized by at least one of the paratopes of the candidate antibody, said second protein being labeled directly or indirectly with the second member of said pair of FRET partners. This first embodiment is particularly preferred as the cell expressing the Fc receptor will in fact “imitate” an effector cell and thus allow valuable information to be obtained regarding the ability of the candidate antibody to bind to this receptor and by extension to recruit an effector cell.

In this embodiment, the Fc receptor is selected from the receptors in Table 1, and is preferably an Fc gamma receptor, and even more preferably the CD16a receptor (FcγRIIIa) or a variant thereof. Several variants of this receptor are known, in particular the natural variants L66H, L66R, G147D, Y158H, F2035, F176V (or F158V in certain publications). In a preferred embodiment, it is the V158 variant (or V176) that is used. In another embodiment it is the F158 variant (or F176).

In another particular embodiment, the candidate antibody comprises two different paratopes, the first paratope being specific for a first epitope and the second paratope being specific for a second epitope, the first cell expresses, in the extracellular portion of its plasma membrane, a first protein that bears said first epitope, said first protein being labeled directly or indirectly with the first member of said pair of FRET partners, and the second cell expresses, in the extracellular portion of its plasma membrane, a second protein that bears said second epitope, said second protein being labeled directly or indirectly with the second member of said pair of FRET partners.

In this embodiment, it is particularly advantageous for the first protein to be a protein present on the surface of effector cells that are not recruited via Fc receptors, in particular the first protein is preferably selected from the following proteins: CD3, CD28.

The method according to the invention offers really interesting applications, since it constitutes a novel approach that may be integrated in studies for determining whether a given antibody may potentially cause a cytotoxic response mediated by an effector cell.

Thus, the invention also relates to a method for determining the cytotoxic character of a candidate antibody, said method consisting of carrying out the method described above with a first cell imitating an effector cell. According to this method, the appearance of a FRET signal in the presence of the candidate antibody is representative of the affinities of this antibody for the target cell and the effector cell, which are generally correlated with its therapeutic efficacy.

“Effector cell” means a cell capable of causing, directly or indirectly, the death of another cell, and according to the invention, a cell “imitating an effector cell” is a cell expressing, on its surface, one or more proteins normally expressed by said effector cells, but not having their cytotoxic effect.

In this embodiment, the first cell expresses, on its surface, one or more proteins normally expressed on the surface of an effector cell selected from the following cells: NK lymphocyte, macrophage, cytotoxic T lymphocyte, helper T cell, or a cell expressing a protein selected from: CD16a, CD3, CD28.

Preferably, the first cell expresses, on its surface, one or more proteins normally expressed by an NK lymphocyte, based on the ADCC mechanism, which is the commonest mode of action of the therapeutic antibodies. In this case and even more preferably, the first cell expresses an Fc receptor as described above, and in particular the CD16a receptor.

Labeling of the Proteins Recognized by the Candidate Antibody

(a) Coupling of the Membrane Proteins Recognized by the Candidate Antibody with a Donor or an Acceptor Indirectly (Noncovalently)

A membrane protein may be coupled with a fluorescent compound that is a member of a pair of FRET partners indirectly via a second protein capable of combining with it, this second protein itself being labeled directly or indirectly with a fluorescent compound. It is known for example that the FcγRIIIA receptor (CD16a) is associated in the cell membrane with the gamma chain of the FcεRI receptor or with the zeta subunit of CD3; the CD16a receptor may thus be labeled indirectly via these gamma or zeta chains, themselves labeled directly with a fluorescent compound. In the same way, if one of the proteins recognized by the candidate antibody is a membrane receptor forming a dimer with another protein, it will be possible to label this other protein. For example, this is illustrated in example 4, in which the HER2 protein is labeled indirectly via HER3, with which it forms a dimer and which is, in its turn, labeled directly with a fluorophore.

As a complete surprise, it was discovered that the labeling of any membrane protein, i.e. not associating particularly with one of the proteins recognized by the candidate antibody, is sufficient for carrying out the method according to the invention, provided that the protein in question is extensively expressed at the level of the plasma membrane. This may be explained by the “fluid mosaic” model constituted by the cell membrane, i.e. by the fact that the membrane proteins move around on the surface of the cells and that a protein that is extensively expressed will at some time or other be located near the protein recognized by the candidate antibody and that a FRET signal will be measurable. This embodiment is illustrated in example 4, in which the HER2 protein is coexpressed with the CXCR4 protein, with which it does not associate, but which is sufficiently overexpressed for a FRET signal to be observed in the presence of the candidate antibody.

A membrane protein may also be coupled with a fluorescent compound via a pair of binding partners, at least one of which is of a protein nature. In this approach, the membrane protein is fused with the binding partner of protein nature by the conventional techniques of molecular biology (construction of an expression vector comprising a nucleotide sequence coding for the membrane protein, fused with that coding for the protein binding partner, and insertion of the expression vector in the cell). The donor or acceptor is conjugated covalently with the other binding partner, which is called a coupling agent here, which will then be added to the extracellular matrix. Recognition of the binding partners allows indirect labeling of the membrane protein with the donor or the acceptor.

As nonlimiting examples of binding partners of this type, we may mention:
The pair consisting of a specific antibody of an epitope naturally present on the membrane protein, labeled with a fluorescent compound, and this epitope. When this method of labeling is used, it should be verified that the binding of this antibody to this epitope does not interfere with the binding of the candidate antibody to this protein, whether via its Fc fragment or via its paratope. Moreover, in this embodiment, use of an antibody whose constant fragment might be recognized by the membrane protein of interest should be avoided.
The pair consisting of the sequence cysteine-cysteine-X-X-cysteine-cysteine (SEQ ID No. 1) in which X is any amino acid and of a bi-arsenic compound. These bi-arsenic compounds may easily be labeled with an organic molecule of the fluorescein or rhodamine type (see B. A. Griffin et al. (1998) Science. 1998, 281, 269-271 and S. A. Adams et al. (2002) 3. Am. Chem. Soc. 2002, 124, 6063-6076 for details on the technology).
The BTX (bungarotoxin) peptide, a compound of 13 amino acids that is recognized by bungarotoxin (BTX), may be coupled to a fluorescent molecule (see C. M. McCann et al. (2005), Biotechnique (2005), 38, 945-952).
The streptavidin (or avidin)/biotin pair: the binding sequence of streptavidin (SBP-Tag) is a sequence formed by 38 amino acids that has a high affinity for biotin and may be labeled beforehand with a donor or an acceptor (see C. M. McCann et al. (2005), Biotechnique (2005), 38, 945-952).
The sequence of the dihydrofolate reductase enzyme of E. coli (eDHFR), which binds specifically, and with high affinity, ligands such as trimethoprim, on which the donor or the acceptor may be grafted by the technology called “Ligand link Universal labeling technology” from the company Active Motif.
The tag/antitag pairs are binding partners frequently used for labeling proteins. The term “tag” denotes a small protein “label” consisting of an amino acid sequence, generally but not necessarily fairly short (less than 15 amino acids), which is fused to the membrane protein that we wish to label. The term “antitag” denotes an antibody binding specifically to said “tag”. In this embodiment, the “antitag” antibody is bound covalently to the donor or to the acceptor. When the antibody thus labeled is added to the extracellular matrix, it binds to the “tag” conjugated to the membrane protein and the “tag/antitag” interaction allows indirect labeling of this protein with the donor or the acceptor. As nonlimiting examples of “tag/antitag” pairs, we may mention the following pairs, the members of which are available commercially: GST/anti-GST antibody in which GST represents glutathione S-transferase or a fragment thereof; 6HIS/anti-6HIS antibody in which 6HIS is a peptide consisting of 6 histidines; Myc/anti-Myc antibody in which Myc is a peptide consisting of amino acids 410-419 of the human Myc protein; FLAG/anti-FLAG antibody in which FLAG is a peptide having the 8 amino acids DYKDDDDK (SEQ ID No. 2); HA/anti-HA antibody in which HA is an epitope of the influenza hemagglutinin, consisting of the 9 amino acids YPYDVPFYA (SEQ ID No. 3). It is clear that the precise nature of the tag is not critical for carrying out the invention.

(b) Coupling of the Membrane Proteins Recognized by the Candidate Antibody with a Donor or an Acceptor Directly (Covalently)

In this approach, the donor or the acceptor is coupled to the membrane protein by a covalent bond; several techniques have been described and the reagents required for carrying them out are available commercially. For this coupling, one of the following techniques may be used:
Formation of a covalent bond at the level of a reactive group present on the membrane protein, in particular at the level of one of the following groups: the terminal amino group, the carboxylate groups of aspartic and glutamic acids, the amine groups of lysines, the guanidine groups of arginines, the thiol groups of cysteines, the phenol groups of tyrosines, the indole rings of tryptophans, the thioether groups of methionines, the imidazole groups of histidines.
These groups that are present on the membrane protein may form a covalent bond with a reactive group carried by the donor or the acceptor. The appropriate reactive groups are known by a person skilled in the art: a donor or acceptor functionalized with a maleimide group will for example be capable of binding covalently to the thiol groups carried by the cysteines of the protein. Moreover, a donor/acceptor bearing an N-hydroxysuccinimide ester will be capable of attaching covalently to an amine of the membrane receptor.

Use of a Suicide Enzyme
“Suicide enzyme” means proteins that have an enzymatic activity modified by specific mutations that endow them with the ability to bind a substrate rapidly and covalently. These enzymes are called “suicide” because each can only bind a single fluorescent molecule, the activity of the enzyme being blocked by the fixation of the substrate. These enzymes consequently constitute a tool of choice for specifically labeling receptors of interest with a ratio of one fluorescent molecule to one protein. In this approach, a suicide enzyme is fused, by the conventional techniques of molecular biology, with the membrane protein—preferably in its N-terminal portion—and the substrate of the enzyme bound covalently to a donor/acceptor is introduced into the extracellular matrix. The enzymatic reaction has the consequence of covalent binding of the substrate labeled with the enzyme, and therefore labeling of the membrane protein with the donor or the acceptor.
We may mention, as nonlimiting examples, the following enzymes:
   the mutants of O6-alkylguanine DNA alkyltransferase (AGT). The enzymes SNAP-tag (Juillerat et al., Chemistry & Biology, Vol.10, 313-317 April 2003) and CLIP-tag (Gautier et al., Chemistry and Biology, 15, 128-136, February 2008) marketed by the company Cisbio Bioassays are mutants of human AGT of which the substrates are, respectively, O⁶-benzylguanine (abbreviated to BG hereinafter) and O₂-benzylcytosine (abbreviated to BC hereinafter). The enzyme N-AGT (Gronemeyer et al. (Protein engineering, design & selection, Vol. 19, no 7, pp 309-3016, 2006)) is another mutant of this enzyme, whose reactivity with O⁶-benzylguanine is better than that of the enzyme SNAP-tag.
   the mutants of a dehalogenase (such as the enzyme HaloTag marketed by Promega) which also generates an enzymatic reaction of the suicide type (see WO2004/072232), some of the substrates of which are compounds of the chloroalkane family, in particular the chloroalkanes comprising the —NH—CH₂CH₂—O—CH₂CH₂—O—(CH₂)₆—Cl unit. In this case, the donor/acceptor will be conjugated to this type of unit.
   the protein ACP (“Acyl Carrier Protein”), an acyl transporting protein, onto which the 4′-phosphopantetheine residue of coenzyme A on a serine of the ACP is transferred, in the presence of phosphopantetheine transferase (N. George et al., Journal of the American Chemical Society 126 (2004) p 8896-8897). When this approach is used for labeling the membrane protein with the donor or the acceptor, it is necessary to add phosphopantetheine transferase to the reaction mixture. The company NEB markets a fragment of ACP under the trade name “ACP-Tag” for labeling proteins.

When this approach is used for labeling a membrane protein of interest, the cells are transfected with an expression plasmid comprising DNA coding for a fusion protein comprising the suicide enzyme and the receptor of interest. This plasmid may also comprise, upstream of the DNA coding for these proteins, DNA coding for a label such as for example the FLAG epitope, the myc epitope, or else that of influenza hemagglutinin (HA). These labels are not essential but facilitate manipulation of the fusion protein for purposes of control or purification. The transfection is performed by conventional techniques, such as electroporation.

To ensure that the fusion protein will be expressed in the cell membrane, it may be useful to include in the expression plasmid, upstream of the sequence coding for the receptor of interest and the suicide enzyme, that coding for a membrane-addressing peptide, such as the T8 signal peptide or the signal peptide of the mGluR5 receptor, the use of which for this purpose is known by a person skilled in the art. Finally, it may also be desirable to ensure that the sequence coding for the receptor of interest does not comprise a native membrane-addressing sequence, which might become the object of post-translational cleavage of the bond between the receptor of interest and the suicide enzyme: if this is the case, it is preferable not to introduce this domain into the expression plasmid.

Finally, when a suicide enzyme is used for labeling a membrane protein with a FRET partner, the invention comprises a preliminary step of transfection of the cells with an expression vector comprising the DNA sequence coding for a fusion protein corresponding to the membrane protein, fused in its N-terminal portion with a suicide enzyme. The techniques of transfection such as electroporation or the use of lipofectamine are known by a person skilled in the art.

Introduction of the substrate of the enzyme conjugated to a FRET partner into the extracellular matrix will have the effect of labeling the receptor of interest with this FRET partner.

The company Cisbio Bioassays markets Tag-Lite® plasmids allowing expression of fusion proteins with the suicide enzymes known under the trade names SNAP-tag®, CLIP-tag® and Halotag®. The DNA sequences coding for the Fc receptors in Table 1 are known and are available in the databases such as GenBank.

Thus, in an embodiment of the invention using suicide enzymes, either the first protein, or the second protein, or the first and the second proteins are labeled directly via a suicide enzyme, i.e. it is or they are expressed in the form of a fusion protein comprising the first or the second protein and the suicide enzyme, and labeling is carried out by bringing the cells expressing these proteins into contact with the substrate of the enzyme, conjugated to one of the FRET partners.

Expression Vectors, Reagent Kit, Cells

The expression vectors for carrying out the invention are plasmids allowing the expression of a fusion protein comprising the DNA sequence of a protein to which the candidate antibody may bind (via its paratopes or its Fc fragment) and the sequence coding for a suicide enzyme, in particular selected from the mutants of dehalogenase, a fragment of the acyl carrier protein or the mutants of O6-alkylguanine DNA alkyltransferase, the latter being preferred. They may be obtained by incorporating the DNA sequence coding for the protein of interest in one of the plasmids marketed by the company Cisbio Bioassays under the names Tag-lite®, SNAP-tag®, CLIP-tag® and Halo-tag®.

The first and second cells for carrying out the invention are cells, notably mammalian cells, that have been transfected stably or transiently with the expression vectors according to the invention. The techniques for inserting expression vectors in the cells, such as electroporation or the use of lipofectamine, are known by a person skilled in the art. In a particularly advantageous aspect, these cells were incubated in the presence of the substrate of the suicide enzyme, conjugated to a member of a pair of FRET partners, and the gene of interest is thus labeled. These cells may be packaged in frozen form to facilitate their storage and distribution to the users.

The invention therefore also relates to kits of reagents for carrying out the method according to the invention. These kits comprise as a minimum the following components, which may be packaged and sold together or else separately:
mammalian cells expressing a first membrane protein fused to a suicide enzyme;
mammalian cells expressing a second membrane protein fused to a suicide enzyme;
the substrates of the suicide enzymes, conjugated to the members of a pair of FRET partners.

These reagents make it possible to label the cells and then carry out the method according to the invention with a candidate antibody able or likely to bind to the first and to the second protein. The suicide enzymes may be identical or different and are preferably selected from: the mutants of dehalogenase, a fragment of the acyl carrier protein or the mutants of O6-alkylguanine DNA alkyltransferase. If the suicide enzymes fused to the first and second proteins are different, labeling of the two cells with the FRET partners will be able to be carried out in one and the same reaction mixture. If the suicide enzymes are the same, then each cell population will be labeled separately.

A particularly advantageous reagent kit consists of supplying prelabeled and frozen cells, which are directly usable for carrying out the method according to the invention with a candidate antibody able or likely to bind to a first and a second protein. Such a kit comprises:
mammalian cells expressing a first membrane protein fused to a suicide enzyme and labeled with the first member of a pair of FRET partners;
mammalian cells expressing a second membrane protein fused to a suicide enzyme and labeled with the second member of a pair of FRET partners.

Pairs of FRET Partners

According to the invention, membrane proteins are labeled with a member of a pair of FRET partners, in particular with a fluorescent energy-donor compound or a fluorescent energy-acceptor compound.

FRET is defined as a nonradiative energy transfer resulting from a dipole-dipole interaction between an energy donor and an energy acceptor. This physical phenomenon requires energetic compatibility between these molecules. This signifies that the emission spectrum of the donor must overlap, at least partially, the absorption spectrum of the acceptor. In agreement with Förster's theory, FRET is a process that depends on the distance separating the two molecules, donor and acceptor: when these molecules are close to one another, a FRET signal will be emitted.

Selection of the pair of donor/acceptor fluorophores for obtaining a FRET signal is within the capability of a person skilled in the art. Donor-acceptor pairs usable for studying the FRET phenomena are notably described in the work of Joseph R. Lakowicz (Principles of fluorescence spectroscopy, 2^{nd} edition 338), to which a person skilled in the art will be able to refer.

The fluorescent energy donor compounds that are long-lived (>0.1 ms, preferably between 0.5 and 6 ms), in particular the chelates or cryptates of rare earths, are advantageous since they make it possible to perform time-resolved measurements, i.e. to measure TR-FRET signals (“Time Resolved FRET”) avoiding much of the background noise emitted by the measurement medium. They are preferred, for this reason and generally, for carrying out the method according to the invention. Advantageously, these compounds are complexes of lanthanides. These complexes (such as chelates or cryptates) are particularly suitable as a member of the pair of energy donor FRET partners.

The complexes of dysprosium (Dy3+), of samarium (Sm3+), of neodymium (Nd3+), of ytterbium (Yb3+) or of erbium (Er3+) are rare earth complexes that are also suitable for the purposes of the invention, but the complexes of europium (Eu3+) and of terbium (Tb3+) are particularly preferred.

Numerous rare earth complexes have been described and several are currently marketed by the companies PerkinElmer, Invitrogen and Cisbio Bioassays.

Examples of chelates or cryptates of rare earths suitable for the purposes of the invention are:
The cryptates of lanthanides, comprising one or more pyridine units. These rare earth cryptates are described for example in patents EP 0 180 492, EP 0 321 353, EP 0 601 113 and in international application WO 01/96 877. The cryptates of terbium (Tb3+) and of europium (Eu3+) are particularly suitable for the purposes of the present invention. Cryptates of lanthanides are marketed by the company Cisbio Bioassays. We may mention, as nonlimiting examples, the cryptates of europium of the following formulas (which may be coupled to the compound to be labeled via a reactive group, here for example an NH₂ group):

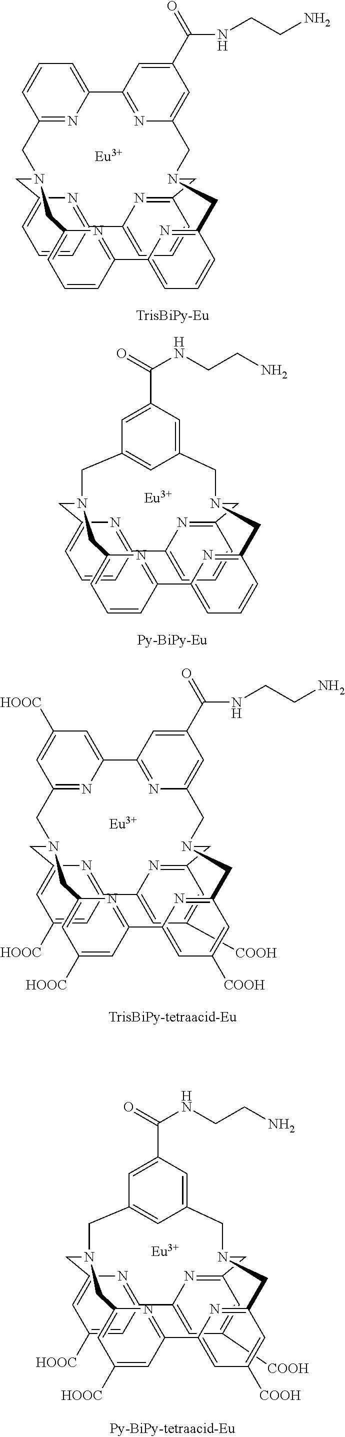

The chelates of lanthanides described notably in patents U.S. Pat. No. 4,761,481, U.S. Pat. No. 5,032,677, U.S. Pat. No. 5,055,578, U.S. Pat. No. 5,106,957, U.S. Pat. No. 5,116,989, U.S. Pat. No. 4,761,481, U.S. Pat. No. 4,801,722, U.S. Pat. No. 4,794,191, U.S. Pat. No. 4,637,988, U.S. Pat. No. 4,670,572, U.S. Pat. No. 4,837,169, U.S. Pat. No. 4,859,777. Patents EP 0 403 593, U.S. Pat. No. 5,324,825, U.S. Pat. No. 5,202,423, U.S. Pat. No. 5,316,909 describe chelates comprising a nonadentate ligand such as terpyridine. Chelates of lanthanides are marketed by the company PerkinElmer.
Complexes of lanthanides comprising a chelating agent, such as tetraazacyclododecane, substituted with a chromophore comprising aromatic rings, such as those described by Poole R. et al. in Biomol. Chem, 2005, 3, 1013-1024 “Synthesis and characterization of highly emissive and kinetically stable lanthanide complexes suitable for usage in cellulo”, may also be used. The complexes described in application WO 2009/10580 may also be used.
The cryptates of lanthanides described in patents EP 1 154 991 and EP 1 154 990 are also usable.
The terbium cryptate of the following formula (which may be coupled to a compound to be labeled via a reactive group, here for example an NH₂ group):

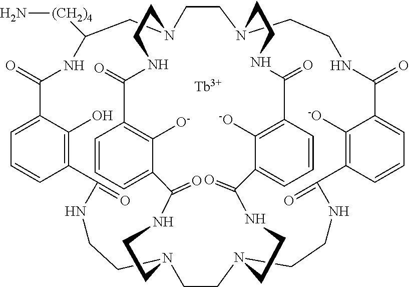

and whose synthesis is described in international application WO 2008/063721 (compound 6a page 89).
The terbium cryptate Lumi4-Tb from the company Lumiphore, marketed by Cisbio Bioassays.
The quantum dye from the company Research Organics, of the following formula (which may be coupled to the compound to be labeled via a reactive group, here NCS):

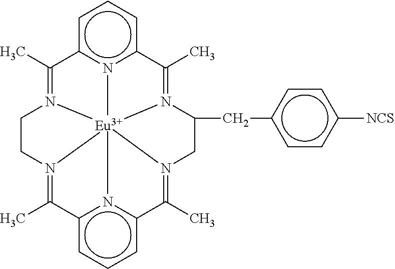

The chelates of ruthenium, in particular the complexes consisting of a ruthenium ion and several bipyridines such as ruthenium(II) tris(2,2′-bipyridine).
The terbium chelate DTPA-cs124 Tb, marketed by the company Life Technologies with the following formula (which may be coupled to the compound to be labeled via a reactive group R) and whose synthesis is described in U.S. Pat. No. 5,622,821.

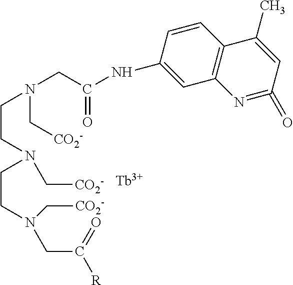

The terbium chelate with the following formula, described by Latva et al. (Journal of Luminescence 75: 149-169):

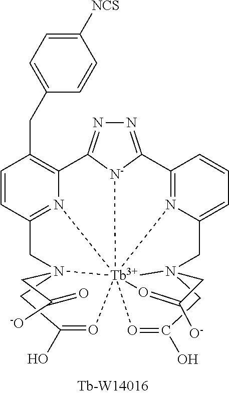

Particularly advantageously, the fluorescent donor compound is selected from: a europium cryptate; a europium chelate; a terbium chelate; a terbium cryptate; a ruthenium chelate; and a quantum dye; the chelates and the cryptates of europium and of terbium being particularly preferred.

The complexes of dysprosium (Dy3+), of samarium (Sm3+), of neodymium (Nd3+), of ytterbium (Yb3+) or of erbium (Er3+) are also complexes of rare earths suitable for the purposes of the invention.

The fluorescent acceptor compounds may be selected from the following group: the allophycocyanins, in particular those known by the trade name XL665; luminescent organic molecules, such as rhodamines, the cyanines (for example Cy5), squaraines, coumarins, proflavins, acridines, fluoresceins, the derivatives of boron-dipyrromethene (marketed under the name “Bodipy”), the fluorophores known by the name “Atto”, the fluorophores known by the name “DY”, the compounds known by the name “Alexa”, nitrobenzoxadiazole. Advantageously, the fluorescent acceptor compounds are selected from allophycocyanins, rhodamines, cyanines, squaraines, coumarins, proflavins, acridines, fluoresceins, the derivatives of boron-dipyrromethene, nitrobenzoxadiazole.

The expressions “the cyanines” and “the rhodamines” are to be understood respectively as “the derivatives of cyanine” and “the derivatives of rhodamine”. A person skilled in the art knows these various fluorophores, which are commercially available.

The “Alexa” compounds are marketed by the company Invitrogen; the “Atto” compounds are marketed by the company Attotec; the “DY” compounds are marketed by the company Dyomics; the “Cy” compounds are marketed by the company Amersham Biosciences; the other compounds are marketed by various suppliers of chemical reagents, such as the companies Sigma, Aldrich or Acros.

The following fluorescent proteins may also be used as fluorescent acceptor compound: the cyan fluorescent proteins (AmCyan1, Midori-Ishi Cyan, mTFP1), the green fluorescent proteins (EGFP, AcGFP, TurboGFP, Emerald, Azami Green, ZsGreen), the yellow fluorescent proteins (EYFP, Topaz, Venus, mCitrine, YPet, PhiYFP, ZsYellow1, mBanana), the orange and red fluorescent proteins (Orange kusibari, mOrange, tdtomato, DsRed, DsRed2, DsRed-Express, DsRed-Monomer, mTangerine, AsRed2, mRFP1, JRed, mCherry, mStrawberry, HcRed1, mRaspberry, HcRed-Tandem, mPlim, AQ143), the fluorescent proteins in the far red (mKate, mKate2, tdKatushka2).

For the purposes of the invention, the derivatives of cyanines or of fluorescein are preferred as fluorescent acceptor compounds.

The invention is illustrated by the following examples, given purely indicatively.

### EXAMPLE 1

### Material and Method

Reagents Used

Culture medium: DMEM/Glutamax+10% FCS+ streptomycin (50 μg/m1), penicillin 50 U/ml, HEPES 2 mM, nonessential amino acids 1% (InVitrogen)

OptiMEM: culture medium used for transfection, Invitrogen

Lipofectamine 2000: Invitrogen

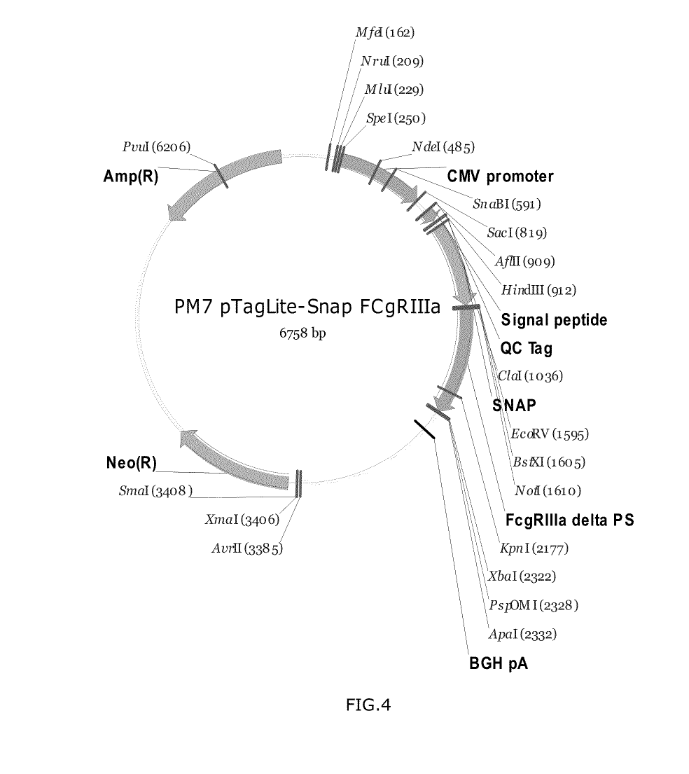

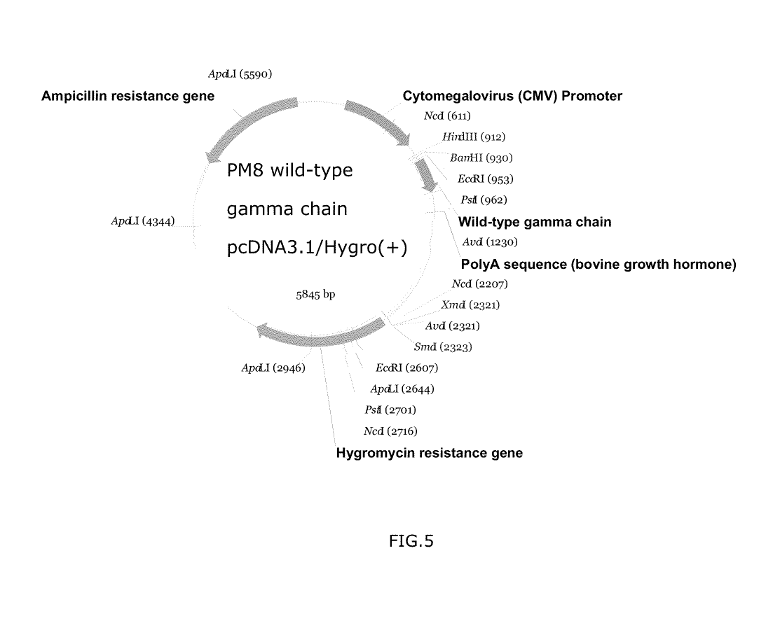

SNAP-CD16A Plasmid (pM7 pTagLite-Snap FcgRIIIa): prepared by inserting the nucleic acid sequence (SEQ ID No. 4) coding for the CD16a receptor (variant V158 (or V176) GenBank ref. NM_{—}000569.6, protein sequence NP_{—}000560.5) in the SNAP-tag® pT8-SNAP-Neomycin plasmid (Cisbio Bioassays), see FIG. 4.

Gamma chain plasmid of the FcεRI receptor (pM8 wild-type gamma chain pcDNA3.1/Hygro(+): DNA sequence (SEQ ID No. 5) coding for the gamma chain of the FcεRI receptor was inserted in an expression plasmid by conventional techniques. The map of this plasmid is shown in FIG. 5. It is known that the CD16a receptor forms dimers with this gamma chain, and it is therefore recommended to coexpress the two proteins. The inventors nevertheless discovered that this coexpression was not necessary for carrying out the method according to the invention, even if it is recommended.

Plasmids Halo-HER1, Halo-HER2 and Halo-HER3: prepared by inserting the nucleic acid sequence coding for the HER1, HER2 or HER3 receptor (protein sequence uniprot ref P00533, P04626, P21860) in the pT8-HaloTag-Neomycin plasmid (Cisbio Bioassays)

Plasmids SNAP-HER2 and SNAP-CXCR4: prepared by inserting the nucleic acid sequence coding for the HER2 or CXCR4 receptor (uniprot ref. P04626 and P61073) in the SNAP-tag® pT8-SNAP-Neomycin plasmid (Cisbio Bioassays)

FCS: fetal calf serum, Invitrogen

DMSO: Dimethylsulfoxide, SIGMA

Tag-lite® SNAP-Lumi4Tb: Donor compound, Cisbio Bioassays, ref SSNPTBX

Tag-lite® HALO-Red: Red acceptor compound, Cisbio Bioassays ref SHALOREDE

Tag-lite® SNAP-Red: Red acceptor compound, Cisbio Bioassays ref SSNPREDE

Tag-lite®: labeling buffer, Cisbio Bioassays, ref LABMED.

CD16/Gamma chain transfection mixture: Mixture consisting on the one hand of 5 μl of “SNAP-CD16A” plasmid (1 μg/μl), 15 μl of Lipofectamine and 2.6 ml of OptiMEM medium and on the other hand of 10 μl of “gamma chain” plasmid (1 μg/μl), 30 μl of Lipofectamine and 5.4 ml of OptiMEM medium

HER1 transfection mixture: Mixture consisting of 20 μl of “Halo-HER1” plasmid (1 μg/μl), 60 μl of Lipofectamine and 8 ml of OptiMEM medium

HER2 transfection mixture: Mixture consisting of 20 μl of “Halo-HER2” plasmid (1 μg/μl), 60 μl of Lipofectamine and 8 ml of OptiMEM medium

HER2 and HER3 transfection mixture: Mixture consisting of 10 μl of “SNAP-HER2” plasmid (1 μg/μl), 30 μl of Lipofectamine and 4 ml of OptiMEM medium and of 10 μl of “Halo-HER3” plasmid (1 μg/μl), 30 μl of Lipofectamine and 4 ml of OptiMEM medium

HER2 and CXCR4 transfection mixture: Mixture consisting of 10 μl of “Halo-HER2” plasmid (1 μg/μl), 30 μl of Lipofectamine and 4 ml of OptiMEM medium and of 10 μl of “SNAP-CXCR4” plasmid (1 μg/μl), 30 μl of Lipofectamine and 4 ml of OptiMEM medium

Cetuximab: specific antibody of HER1

Herceptin: specific antibody of HER2

Pertuzumab: specific antibody of HER2

Transfection of the Cells

The transfection mixture (CD16/Gamma chain, HER1, HER2, HER2/HER3 or HER2/CXCR4) was incubated for 20 minutes at room temperature.

HEK293 cells were cultured in a T175 flask. Once these cells reached 60 to 70% confluence, the culture medium was removed and the cells were washed with 10 ml of PBS medium. 8 ml of the transfection mixture was then added to these cells, as well as 12 ml of culture medium. The cells were then incubated overnight at 37° C.

Labeling the Cells with the Fluorescent Compounds

After removal of the transfection mixture and washing with 10 ml of PBS, 10 ml of one of the following fluorescent compounds was added:
Tag-lite® SNAP-Lumi4-Tb (donor compound, 100 nM) in solution in the Tag-lite® labeling buffer for the cells transfected with the CD16a/gamma chain transfection mixture.
Tag-lite® HALO-Red (acceptor compound, 100 nM) in solution in the Tag-lite® labeling buffer for the cells transfected with the HER1, HER2 and HER2/HER3 transfection mixtures.
Tag-lite® SNAP-Red (acceptor compound, 100 nM) in solution in the Tag-lite® labeling buffer for the cells transfected with the HER2/CXCR4 transfection mixtures.

After incubation for 1 h at 37° C., the mixture was washed 4 times with the Tag-lite® labeling buffer, then 5 ml of “cell dissociation buffer” (Sigma) was added to dissociate the cells and 5 ml of OptiMEM medium. The cells thus obtained were centrifuged for 5 min at 1200 rev/min. The pellet was then resuspended with 1 ml+1 ml of Tag-lite® labeling buffer so as to be able to count the cells.

This suspension was re-centrifuged for 5 min at 1200 rev/min and the pellet was taken up in culture medium containing 10% FCS and 10% DMSO to obtain a suspension of labeled cells at a concentration of 1 million cells/ml.

This suspension was divided into aliquots in tubes at a rate of 1 ml per tube, and the tubes were placed in a Nalgene dish at −80° C.

### EXAMPLE 2

### FRET HEK-CD16a and HEK-HER1

The cells expressing the HEK-CD16a and HEK-HER1 proteins, prepared and labeled with fluorescent compounds in example 1, were thawed at 37° C. and quickly mixed with 15 ml of PBS. The suspension obtained was centrifuged for 5 min at 1200 rev/min and the supernatant was removed. The pellet was re-suspended in Tag-lite® buffer to obtain a cellular suspension. The HEK-CD16a and HEK-HER1 cells were distributed in the wells of a multipoint 384 LV plate at the respective concentrations of 10 000 cells per well under 5 μl and 20 000 cells per well under 5 μl.

An antibody (Cetuximab, Herceptin, Pertuzumab or any human antibody of isotype IgG1 as negative control) was added to different final concentrations of 0.01 nM to 100 nM under 10 μl. The 384 plate was read immediately, then after 30 min, 1 h, 2 h30, 3 h40, 5 h, 6 h and 12 h.

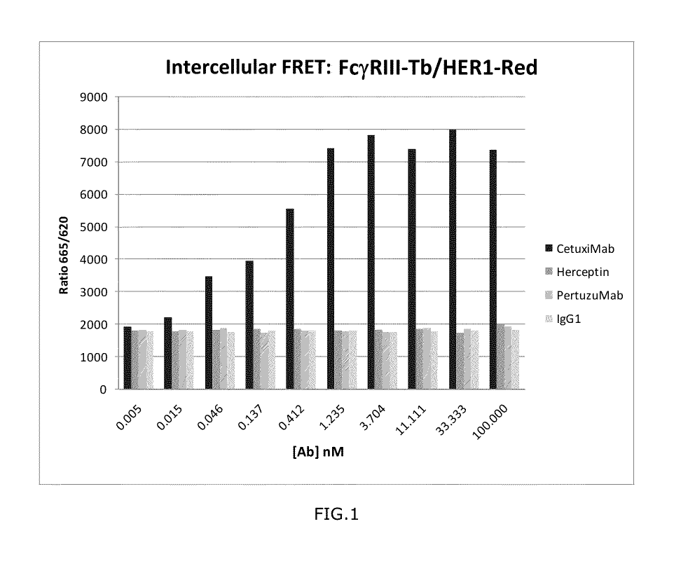

The results presented in FIG. 1 show that only the Cetuximab antibody, which is known to bind both to CD16a via its Fc fragment and to HER1 via its variable domains, causes appearance of a FRET signal. This signal results from bringing the cells expressing CD16a and those expressing HER1 closer together and shows that the method according to the invention makes it possible to determine the ability of an antibody to keep two cells close to one another, moreover quantitatively.

### EXAMPLE 3

### FRET HEK-CD16a and HEK-HER2

The same experiment as in example 2 was carried out but this time with HEK-HER-2 cells in place of the HEK-HER-1 cells.

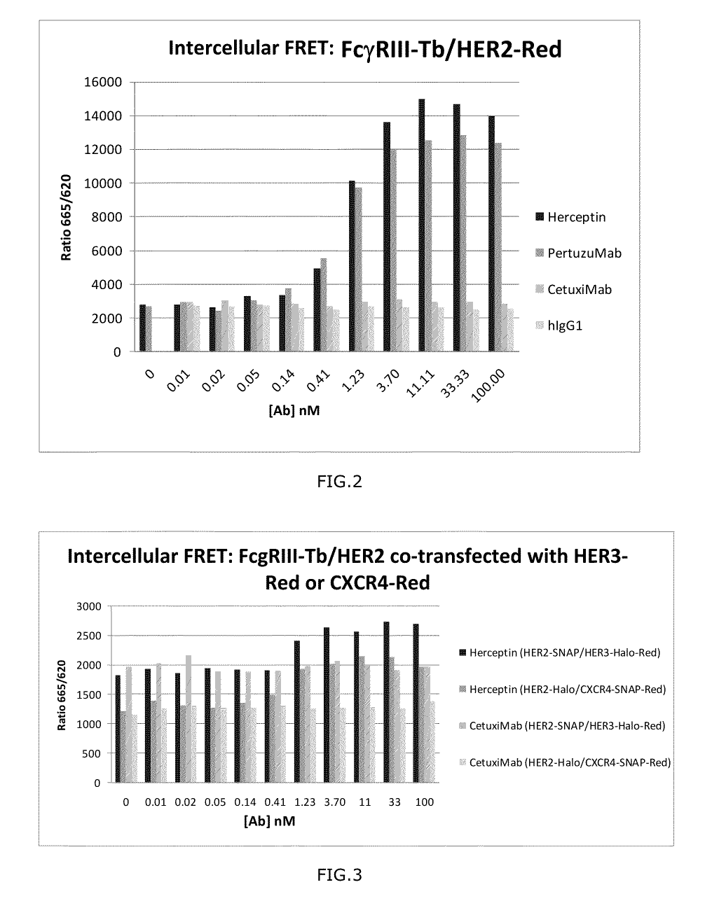

The results presented in FIG. 2 show once again that the method according to the invention makes it possible to distinguish the antibodies binding both to the CD16a receptor and to HER2 (Herceptin, pertuzumab), from the antibodies only binding to the CD16a receptor (Cetuximab, hIgG1 Antibody).

### EXAMPLE 4

### FRET HEK-CD16a and HEK-HER2-HER3 or HEK-HER2-CXCR4, Indirect Labeling

The same experiment as in example 2 was carried out but this time with HEK-HER2-HER3 cells labeled on HER3 or HEK-HER2-CXCR4 labeled on CXCR4, in place of the HEK-HER-1 cells. This experiment aims to show that the method according to the invention may be carried out with indirect labeling of one of the proteins recognized by the candidate antibody, here indirect labeling of HER2 with HER3 or CXCR4.

The results presented in FIG. 3 show once again that the method according to the invention makes it possible to distinguish the antibodies binding both to the CD16a receptor and to HER2 (Herceptin), from the antibodies only binding to the CD16a receptor (Cetuximab), even if the signal observed here is lower than in the preceding examples.

1. A method for determining the ability of a candidate antibody to keep a first cell and a second cell close to one another, this method comprising the following steps:
bringing the following elements in contact:
(i) a first cell expressing, in the extracellular portion of its plasma membrane, a first protein that is known to be or is suspected of being recognized by the candidate antibody, said first protein being labeled directly or indirectly with the first member of a pair of FRET partners,
(ii) a second cell expressing, in the extracellular portion of the plasma membrane, a second protein that is also known to be or suspected of being recognized by the candidate antibody, said second protein being labeled directly or indirectly with the second member of said pair of FRET partners,
(iii) said candidate antibody;
measuring of the FRET signal and comparison with the signal measured in the absence of the candidate antibody, wherein an increase in the signal measured in the presence of the candidate antibody relative to that measured in its absence is indicative of the ability of said antibody to keep the first cell and the second cell close to one another.

2. The method as claimed in claim 1, wherein said first protein is a membrane receptor of the Fc fragment of the antibodies, and wherein said second protein bears an epitope recognized by at least one of the paratopes of the candidate antibody.

3. The method as claimed in claim 2, wherein the Fc receptor is an Fc gamma receptor.

4. The method as claimed in claim 2, wherein the Fc receptor is the CD16a receptor or a variant thereof.

5. The method as claimed in claim 1, wherein the candidate antibody comprises two different paratopes, the first paratope being specific for a first epitope and the second paratope being specific for a second epitope, in that said first protein bears said first epitope, and wherein said second protein bears said second epitope.

6. The method as claimed in claim 5, wherein said first protein is selected from: CD3, CD28.

7-8. (canceled)

9. The method as claimed in claim 1, wherein either the first protein, or the second protein, or the first and the second proteins are labeled directly via a suicide enzyme, and wherein the labeling is carried out by bringing the cells expressing these proteins into contact with the substrate of the enzyme, conjugated to one member of the pair of FRET partners.

10. The method as claimed in claim 9, wherein the suicide enzyme is selected from: the mutants of O6-alkylguanine DNA alkyltransferase, the mutants of dehalogenase, and a fragment of the acyl carrier protein.

11. A reagent kit for carrying out the method as claimed in claim 1, which comprises the following components:
mammalian cells expressing a first membrane protein fused to a suicide enzyme;
mammalian cells expressing a second membrane protein fused to a suicide enzyme;
the substrates of the suicide enzymes, conjugated to the members of a pair of FRET partners.

12. A reagent kit for carrying out the method as claimed in claim 1, which comprises the following components:
mammalian cells expressing a first membrane protein fused to a suicide enzyme and labeled with the first member of a pair of FRET partners;
mammalian cells expressing a second membrane protein fused to a suicide enzyme and labeled with the second member of a pair of FRET partners.

13. The method as claimed in claim 3, wherein the Fc receptor is the CD16a receptor or a variant thereof.

14. A method for determining the cytotoxic character of a candidate antibody, which method comprises the steps of:
bringing the following elements in contact:
(i) a first cell expressing, in the extracellular portion of its plasma membrane, a first protein that is known to be or is suspected of being recognized by the candidate antibody, said first protein being labeled directly or indirectly with the first member of a pair of FRET partners, wherein said first cell is a cell imitating an effector cell selected from: NK lymphocytes, macrophages, cytotoxic T lymphocytes, and helper T cells;
(ii) a second cell expressing, in the extracellular portion of the plasma membrane, a second protein that is also known to be or suspected of being recognized by the candidate antibody, said second protein being labeled directly or indirectly with the second member of said pair of FRET partners;
(iii) said candidate antibody;
measuring the FRET signal, wherein the appearance of a FRET signal is representative of the affinities of the candidate antibody for the first and the second cells.

15. The method as claimed in claim 14, wherein said first protein is a membrane receptor of the Fc fragment of the antibodies, and wherein said second protein bears an epitope recognized by at least one of the paratopes of the candidate antibody.

16. The method as claimed in claim 15, wherein the Fc receptor is an Fc gamma receptor.

17. The method as claimed in claim 14, wherein the candidate antibody comprises two different paratopes, the first paratope being specific for a first epitope and the second paratope being specific for a second epitope, wherein said first protein bears said first epitope, and wherein said second protein bears said second epitope.

18. A method for determining the cytotoxic character of a candidate antibody, which method comprises the steps of:
bringing the following elements in contact:
(iv) a first cell expressing, in the extracellular portion of its plasma membrane, a first protein that is known to be or is suspected of being recognized by the candidate antibody, said first protein being labeled directly or indirectly with the first member of a pair of FRET partners, wherein said first protein is selected from: CD16a, CD3, and CD28;
(v) a second cell expressing, in the extracellular portion of the plasma membrane, a second protein that is also known to be or suspected of being recognized by the candidate antibody, said second protein being labeled directly or indirectly with the second member of said pair of FRET partners;
(vi) said candidate antibody;
measuring the FRET signal, wherein the appearance of a FRET signal is representative of the affinities of the candidate antibody for the first and the second cells.

19. The method as claimed in claim 18, wherein said first protein is CD16a.
